(19) United States
(12) Patent Application Publication
(10) Pub. No.: US 2013/0138130 A1
(43) Pub. Date: May 30, 2013

(54) Tatooing Machine and Tattooing Machine Armature Assembly

(71) Applicant: Sergei Nikolaevich Nizov, Orel (RU)

(72) Inventor: Sergei Nikolaevich Nizov, Orel (RU)

(21) Appl. No.: 13/696,018

(22) PCT Filed: May 5, 2011
(86) PCT No.: PCT/RU2011/000305

§ 371 Date: Jan. 11, 2013

(30) Foreign Application Priority Data
May 5, 2010 (RU) 2010117602
Feb. 17, 2011 (RU) 2011106215

(51) Int. Cl. A61M 37/00

(52) U.S. Cl. CPC A61M 37/0076
USPC 606/186

(57) ABSTRACT

A tattooing machine armature assembly is provided, said tattooing machine armature assembly comprising an armature, an armature spring, a contact spring, and a locking screw, wherein the armature is mounted on the armature spring by means of the locking screw so as to oscillate, wherein the armature is embodied with a hook while the contact spring is mounted so as to cooperate with the hook of the armature. A tattooing machine is provided as well, said tattooing machine comprising a frame, a vise, one inductance coil as a minimum, a magnetic circuit, an armature, an armature spring, a contact spring, a capacitor, a bar, contact islands, locking screws, and insulation spacers, wherein the armature is mounted on the frame by means of the armature spring so as to oscillate when subjected to the magnetic field produced by the inductance coils while the bar and one of contact islands are electrically insulated from the frame, said tattooing machine comprising a stop mounted so as to intercept the contact spring at closure of the electric circuit of the apparatus while the contact spring is mounted to be pre-compressed.

### FIELD OF THE INVENTION

A tattooing machine can be used to tattoo humans and animals with artistic and episematic tattoos and also to administer vaccine matters and pharmaceuticals intradermally.

### PRIOR ART

Technical solutions are known from the prior art which are similar to the present invention, for example, such as “Contact bar assembly for a tattooing device” disclosed in U.S. Pat. No. 6,282,987 B1 of Sep. 4, 2001. The present design is a classic induction tattooing machine and structurally is the closest one to the present invention. The machine comprises a frame, a vise, one electromagnetic coil as a minimum, a magnetic circuit, an armature, a spring armature reed, a contact spring, a capacitor, a bar, contact points, locking screws and insulation spacers, said armature being mounted on the frame by means of the spring armature reed so as to oscillate when subjected to the magnetic field produced by the inductance coils while the bar and one of contact points are electrically insulated from the frame.

The advantages of the present tattooing machine is the relative structural simplicity, the reliability and durability, the high productivity of the tattooing process while low-frequency and non-harmonic oscillations of the impact power represent one of the main disadvantages; they are stronger expressed in mass-produced machines and in machines having a lighter frame as well, and are weaker expressed in customized machines manufactured manually and having a relative heavier frame. In most cases it turns out that oscillations of the impact power in machines of the middle price group are conspicuous aurally under idle conditions.

The second disadvantage of the present device is contact sparking which results in quick erosion of contact surfaces and therefore in destabilization of the machine operation.

A tattooing machine is also known as disclosed in U.S. Pat. No. 4,159,659 of Jul. 3, 1979. The present machine comprises a frame, a clip, electromagnet coils with cores, an armature, an armature spring, a contact spring, a contact screw, screw fastener means and switch contacts. On the principle of its constitution, the present machine is no different from the design described above, and its main distinction consists in a frame embodied on the basis of two side plates and three spools, wherein one spool serves to fasten the clip and electromagnet coils, the second spool serves to mount the armature spring and the third spool serves to mount a contact screw. Due to elongated slots formed in the side plates of the frame, the design of the machine allows many variants for spools to move. In intention of the inventor, such movements should assists in ideal setting of the device into a maximally stable mode of operation which most likely was not practiced just for once because of absolute impossibility to understand the influence pattern of such a number of adjustments upon the operation of the device.

At the same time, it was succeeded to clear during long experiments and theoretical studies that the instability in the operation mode inherent in all induction tattooing machines of the classic type without distinction is caused exclusively by oscillations of the upper end of the contact spring relative to the armature.

Oscillation superposition of said oscillations and oscillations of the armature relative to the frame, said armature being sprung by the armature spring on the frame, results in that the upper end of the contact spring non-harmonically oscillates relative to the frame, which in turn results in phase mismatch of the armature and electric pulses supplied to the inductance coils. In other words, closure of the electrical circuit in the classic tattooing machine takes place in different positions of the armature, but this inevitably causes instability in the operation mode of the machine. Use of a wedged rubber ring between the armature and the contact spring solves this problem only partially and thereby limits the setting of the stable mode to infinite searches for an optimum relationship of the armature spring length and stiffness, the contact spring length and stiffness, the striker mass, the rubber ring diameter and stiffness, etc.

In addition, absence of pre-compression of the contact spring also gives rise to the problem of contact sparking and eroding, because the increase of an air gap becomes slower when the contact is open since the straighten contact spring makes a series of decaying harmonic oscillations after loss of contact, the first oscillation being directed towards the contact screw and makes the divergence of contact surfaces slower. Apart from additional phase mismatch, this effect results in firing of an electric arc in a zone where the electric arc contacts and the fast erosion of contact surfaces take place. Sometimes, the erosion goes so intensively that the finest particles of a brassy contact screw are visible which fly out of a zone where the contact operates.

### SUMMARY OF THE INVENTION

On the basis of conclusions made in the course of studying disadvantages of classical designs, the main problem to be solved by the present invention was to achieve a maximum phase synchronism in opening and closing a contact pair of the tattooing machine relative to an armature position, and also to provide a maximally fast increase of an air gap between contact surfaces at closure thereof.

It is an object of the invention to improve consumer properties of the tattoo machine due to improvement in the stability of its operation, simplification of adjustment operations, and improvement in the durability of basic parts.

In order to achieve said object, in the prior art design of a tattooing machine comprising a frame, a vise, one inductance coil as a minimum, a magnetic circuit, an armature, an armature spring, a contact spring, a capacitor, a bar, contact islands, locking screws and insulation spacers, said armature being mounted on the frame by means of the armature spring so as to oscillate when subjected to the magnetic field produced by the inductance coils while the bar and one of contact islands are electrically insulated from the frame, there are structural changes as follows: the machine comprises a stop mounted so as to intercept the contact spring in closure of an electric circuit of the apparatus while the contact spring is mounted to be pre-compressed.

Further, the machine comprises a contact screw mounted on the bar so as to cooperate with the contact spring, the stop is embodied as an armature hook, and the contact spring is mounted on the armature and pre-compressed due to engagement with the armature hook.

Further, the machine comprises a contact and an adjustment screw, the stop is embodied as an adjustment screw and mounted on the bar, the contact spring is mounted on the bar and electrically insulated from the armature spring while the contact is mounted at an upper part of the armature so as to cooperate with the contact spring.

Because of said structural changes, the operation stability of the tattooing machine becomes better and its adjustment becomes simpler while its service life becomes longer.

### BRIEF DESCRIPTION OF DRAWINGS

The apparatus of the invention is illustrated by Figures of the drawings, wherein:

FIG. 1 is a rear view of an armature assembly;

FIG. 2 is a side view of the armature assembly;

FIG. 3 is a side view of a tattooing machine in a middle position of an armature;

FIG. 4 is a side view of the tattooing machine in a front position of the armature;

FIG. 5 is a side view of the tattooing machine in a rear position of the armature;

FIG. 6 is a sectional view of contact islands of the tattooing machine;

FIG. 7 is a side view of an embodiment of the tattooing machine;

FIG. 8 is a cross-sectional view of a contact screw locking assembly in an embodiment of the tattooing machine;

FIG. 9 is a cross-sectional view of the armature assembly in an embodiment of the tattooing machine;

FIG. 10 is an embodiment of the tattooing machine, wherein the armature is in the middle position while a contact is closed;

FIG. 11 is an embodiment of the tattooing machine, wherein the armature is in the front position while the contact is open;

FIG. 12 is an embodiment of the tattooing machine, wherein the armature is in the rear position while the contact is closed;

FIG. 13 is an embodiment of the tattooing machine, wherein the armature is in the middle position while the contact is closed;

FIG. 14 is an embodiment of the tattooing machine, wherein the armature is in the front position while the contact is open;

FIG. 15 is an embodiment of the tattooing machine, wherein the armature is in the rear position while the contact is closed.

### DESCRIPTION OF EMBODIMENTS OF THE INVENTION

A tattooing machine comprises a frame (1), a bar (2), a vise (3), inductance coils (4), a magnetic circuit (5), an armature (6) with a hook (7), an armature spring (8), a contact spring (9), a capacitor (10), contact islands (11), a contact screw (12), and locking screws (13) and insulation spacers (14). At the same time, the magnetic circuit (5), the bar (2), the armature spring (8), and the contact islands (11) are mounted on the frame (1) while one of the contact islands (11) and the bar (2) are insulated from the frame (1) by means of the insulation spacers (14). The armature (6) is mounted on the armature spring (8) so as to oscillate while the contact spring (9) is mounted on the armature (6) to be pre-compressed as provided by bringing an upper end of the contact spring (9) under the hook (7) of the armature (6). The contact screw (12) is mounted on the bar (2) so as to cooperate with the contact spring (9). The vise (3) serves to fix a tipped handle necessary to hold the machine in progress of operation therewith.

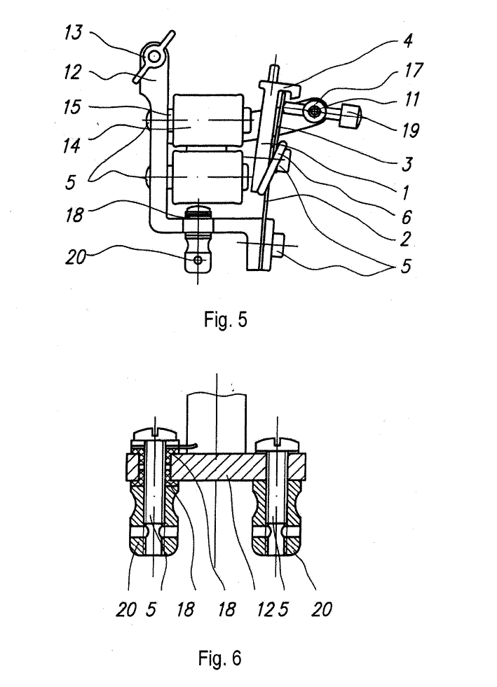

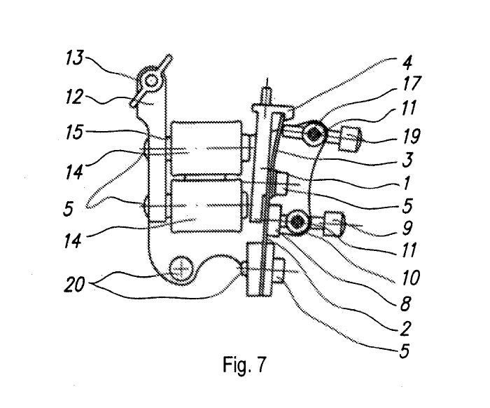

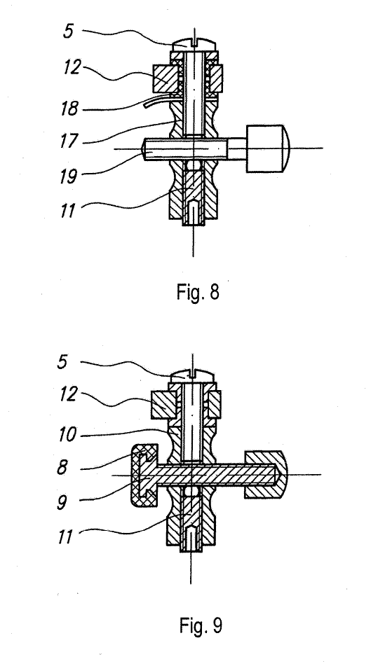

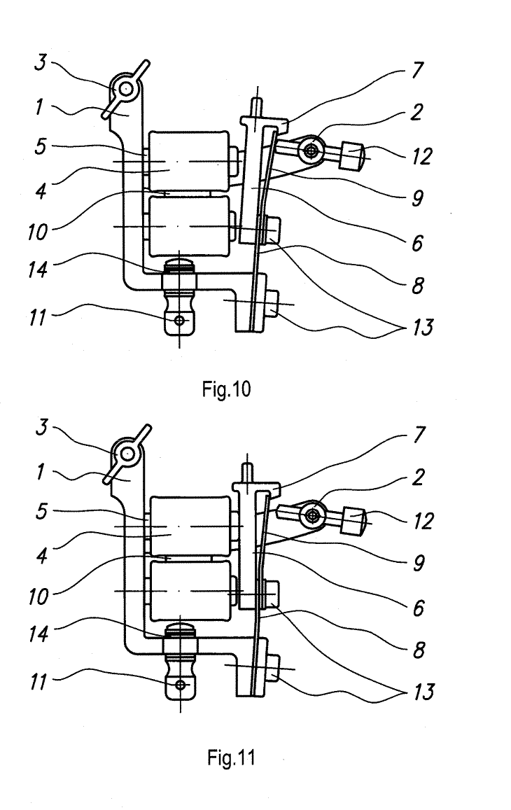

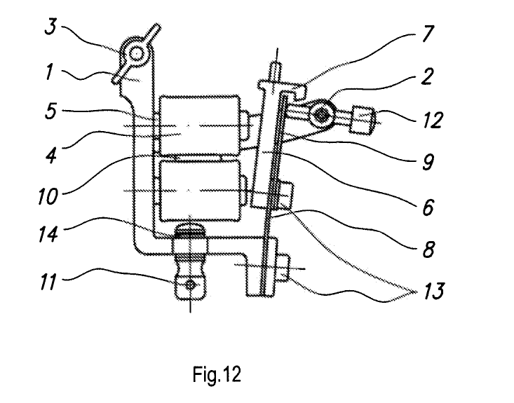

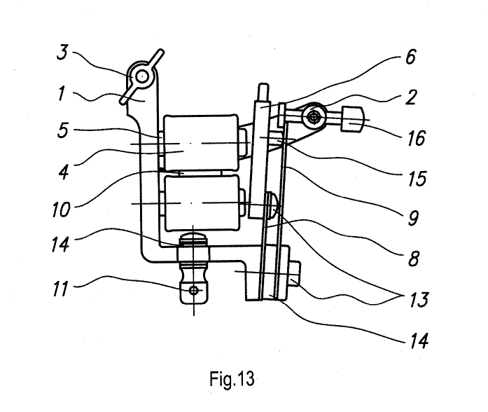

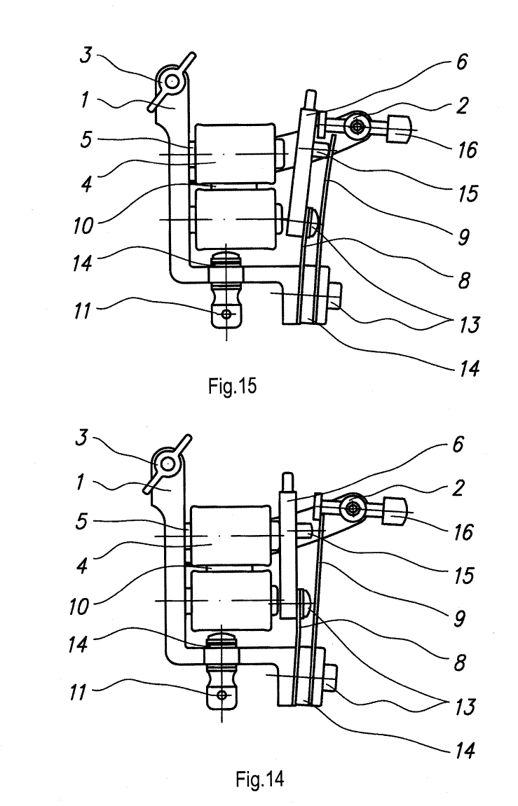

The tattooing machine according to FIGS. 4, 5, 6, 7 is different from the design described above in that the contact spring (9) is mounted on the frame (1) in parallel with the armature spring (8) and is insulated therefrom by means of the insulation spacer (14). In addition, a contact (15) is mounted on the armature (6) so as to cooperate with the contact spring (9) while an adjustment screw (16) is mounted on the bar (2), said screw providing pre-compression of the contact spring (9).

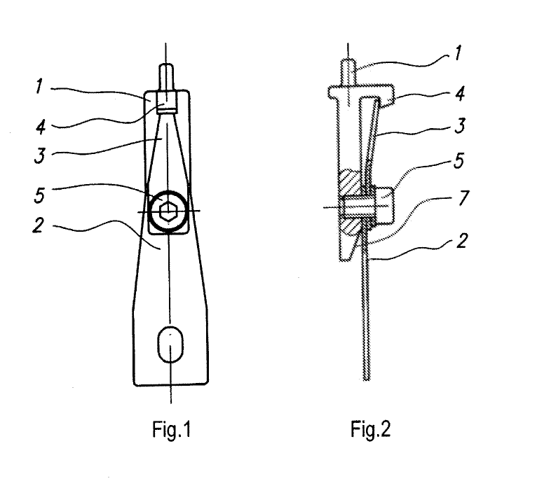

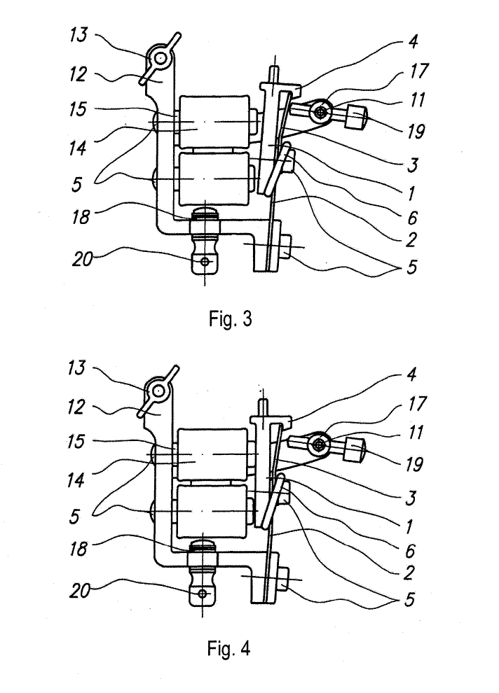

The tattooing machine according to FIGS. 1, 3, 4, 7 operates as follows. In the initial position, the electrical circuit of the machine is closed because the contact screw (12) abuts the contact spring (9).

When a constant voltage is supplied from a power supply to the contact islands (11) of the tattooing machine, an electric current flows through windings of the inductance coils (4) to induce the magnetic field which attracts the armature (6) to the magnetic circuit (5) and bends the armature spring (8). In doing so, as soon as the contact screw (12) discontinues its contact with the contact spring (9), the upper end of the latter is immediately intercepted by the hook (7) of the armature (6). It should be also noted that the contact spring (9) is brought under the hook (7) at a large pre-compression, and when the contact spring (9) is intercepted by the hook (7), therefore, natural oscillations of the contact spring (9) relative to the armature (5) are stopped very quickly, and the armature (6) assembled with the contact spring (9) oscillates on the armature spring (8) as an entire, monolithic structure. Having achieved the extreme front position and having stricken (in some operation modes) at the magnetic circuit (5), the armature (6) begins the rearward motion under the action of the au nature spring (8). When the armature (6) gathers a speed close to a maximum speed in the vicinity of a midpoint of oscillations, the contact spring (9) encounters the contact screw (12), which results in repeated closure of the electrical circuit and in occurrence of the magnetic field which begins brake the armature (6) along with the newly-bent an nature spring (8). Having depleted the kinetic energy resources, the armature (6) stops and begins to move forward to a point of opening the electrical contact. Then, the cycle is repeated until the constant voltage supply to the contact islands (11) is cut off.

At the same time, since the interception of the contact spring (9) by the hook (7) takes place at the maximum speed of the armature (6), the grow of the air gap between contact surfaces is as quick as the electric arcc within the range of operation voltages does not occur, which in turn drastically decreases the erosion of contacts. In practice, the spark is observed between contacts in the prototype of the inventive machine only at a moment of starting the apparatus, until the armature (6) does not get necessary oscillation amplitude, and the machine operates at normal oscillation amplitude of the armature (6) at a voltage of from 4 to 12 volt without a visible sparking.

In addition, the sharp interception of the contact spring (9) by the hook (7) and the stable position of the contact spring (9) relative to the armature (6) at closure of contacts provide the exclusively accurate phase synchronism of an armature (6) position and an electric pulse supplied to the inductance coils (4), which enables operation of the machine in a faultless even and smooth manner irrespectively of values of a voltage supplied to the windings, and of a mechanical load applied to the armature 6.

The tattooing machine according to FIGS. 4, 5, 6, 7 operates as described above, except that the contact spring (9) and the adjustment screw (16)—which fulfills the function of a stop—are arranged on the frame (1) and the bar (2), respectively.

Because of being based on a stationary part of the machine, the contact spring (9)—contrary to the first embodiment of the invention described above—is not subjected to inertial loads when the armature (6) and the magnetic circuit (5) collide, which additionally improves the stability of the apparatus in operation, said stability being already high extremely. The more complicated design and less esthetical appearance should be supposed as the disadvantage in this implementation variant of the machine.

In any event and irrespectively of an embodiment of the present invention, inclusion of said technical features in the prior art design of the tattooing machine drastically improves the stability of its operation, simplifies its adjustment and tuning, elongates the service life of parts.

1. A tattooing machine armature assembly comprising an armature, an armature spring, and a contact spring, wherein the armature is mounted on the armature spring so as to oscillate, said tattooing machine armature assembly being characterized in that the armature is embodied with a hook while an upper end of the contact spring is brought under the hook of the armature.

2. The tattooing machine armature assembly according to claim 1, characterized in that it comprises a locking screw and an elastic ring, the armature is embodied with a bevel and is attached on the armature spring by means of the locking screw while the elastic ring is inserted into a wedge gap between the armature spring and the armature bevel and is fixed in a tensioned state by means of the locking screw.

3. The tattooing machine armature assembly according to claim 1, characterized in that it comprises an elastic cap, an adjustment screw, an adjustment screw bar, and a set screw, the elastic cap is mounted on the adjustment screw so as to cooperate with the armature spring while the adjustment screw and the set screw are mounted on the adjustment screw bar.

4. A tattooing machine comprising:
a frame;
a vise mounted on the frame;
at least one inductance coil mounted on the frame;
a magnetic circuit mounted on the frame under the at least one inductance coil;
a capacitor coupled in parallel with the at least one inductance coil;
a bar attached on the frame through insulation spacers;
a contact screw mounted on the bar;
a contact island mounted on the frame through insulation spacers;
spring mounted on the bar;
wherein the capacitor and the at least one inductance coil are coupled in parallel to the bar and the contact island to form a closed loop,
wherein the tattooing machine comprises the armature assembly according to claim 1, said armature assembly comprising an armature, an armature spring, and a contact spring, wherein the armature is mounted on the frame by means of the armature spring so as to oscillate, and said oscillation is provided by the magnetic field produced by the at least one inductance coil while the contact screw is mounted on the bar so as to cooperate with the contact spring.

5.-6. (canceled)
